(19) United States
(12) Patent Application Publication
(10) Pub. No.: US 2008/0262287 A1
(43) Pub. Date: Oct. 23, 2008

(54) System for delivery of magnetic stimulation

(75) Inventor: Alain Dussau, Campbell, CA (US)

Correspondence Address: TIPS GROUP;c/o Intellevate LLC, P. O. BOX 52050, Minneapolis, MN, 52050, US

(73) Assignee: ETIS Investments, Inc.

(21) Appl. No.: 12/082,854

(22) Filed: Apr. 14, 2008

Related U.S. Application Data
(60) Provisional application No. 60/911,876, filed on Apr. 14, 2007.
(60) Provisional application No. 60/953,467, filed on Aug. 2, 2007.
(60) Provisional application No. 60/953,468, filed on Aug. 2, 2007.

(51) Int. Cl. A61N 2/02

(52) U.S. Cl. 600/13

(57) ABSTRACT

A system for delivering magnetic stimulation to a subject that has a magnetic stimulation coil, a power source, and a patient-positioning device is provided. The magnetic stimulation coil is movably connected to patient-positioning device for fixably positioning the magnetic stimulation coil to stimulate a region of a subject and electrically connected to the power source. A cooling system maintains the magnetic stimulation coil between a temperature of about 5° C. and about 40° C. during operation of the coil for a period of at least 90 minutes. An attachment site for a second magnetic stimulation coil is provided, and the second magnetic stimulation coil is optionally movably connected to the patient-positioning device for positioning the second magnetic stimulation coil independently of the first magnetic stimulation coil to provide a combination delivery of magnetic stimulation to a second region of the subject.

### CROSS-REFERENCE TO RELATED APPLICATIONS

This application claims the benefit of U.S. Provisional Application No. 60/911,876, filed Apr. 14, 2007, U.S. Provisional Application No. 60/953,467, filed Aug. 2, 2007, and U.S. Provisional Application No. 60/953,468, filed Aug. 2, 2007, each of which is hereby incorporated herein by reference in its entirety.

### BACKGROUND

1. Field of the Invention

The teachings provided herein are directed to a system for delivering a magnetic stimulation to a subject, wherein a force field generated by a magnet is applied to the subject for therapeutic purposes.

2. Description of Related Art

“Magnetic stimulation” is a therapeutic procedure that can be used to stimulate neural structures, wherein a force field generated by a magnetic stimulation coil is applied to the subject for therapeutic purposes. The stimulating coil is placed near the intended site of stimulation and trigger pulses initiate brief magnetic pulses. The magnetic fields can pass through clothing, tissue and bone to reach otherwise inaccessible areas, and it operates without stimulating pain fibers at the skin surface.

Magnetic nerve stimulators typically consist of two distinct parts: a high current pulse generator producing discharge currents of 5000 amps or more, and a stimulating coil producing magnetic pulses with field strengths of 1 Tesla or more with a pulse duration of millisecond increments. The magnetic fields produced by many currently available magnetic stimulators are in the range of about 0.5 Tesla to about 3.5 Tesla.

Clinical applications include, but are not limited to, stimulation of the peripheral and central motor pathways, stimulation of the left and right prefrontal cortex, visual cortex, language centre, cerebellum and peripheral sensory nerves, as well as the diagnosis, prognosis, and monitoring of neural conditions. The therapy has applications in psychiatry, cognitive neuroscience, neurology, neurophysiology, and rehabilitation, to name a few. For example, depression has been treated successfully using this technique, which is often referred to as “off-label depression therapy,” as its not yet been approved in the U.S. Use of the magnetic stimulation therapy to treat the brain can be referred to as “transcranial magnetic stimulation”, and an emerging use of transcranial magnetic stimulation includes repetitive transcranial magnetic stimulation.

Magnetic stimulation treatment offers much hope in a variety of ailments including, for example, treatment-resistant depression. Treatment-resistant depression is represented by about 20% of all patients with unipolar depression, which affects 1.5% of the general population, 4,267,500 people in the United States, and 91,500,000 people in the world. Treatment-resistant depression is also represented by about 20% of all patients with bipolar depression, and the number of people with bipolar depression is about half the size of those with unipolar depression.

Magnetic stimulation therapy is still in its infancy. Medical professionals currently hold the coil over the patient's head for the treatment duration, which in many of today's protocols requires that the magnetic stimulation coil be held for at least 30 minutes. At best, the magnetic stimulation equipment can be wheeled over to the patient on a separate cart that may or may not complement the support surface upon which the patient is positioned.

The equipment currently used to administer magnetic stimulation therapy is loud and disturbing to patients and medical professionals. And, the treatment times required are not readily supported by existing equipment, as the currently available magnetic stimulation coils get overheated easily and do not have cooling systems that have been accepted by the medical community as sufficient to meet their ongoing treatment needs. The current equipment does not provide a combination of a means to support a patient during the long treatment times. Moreover, the currently available means do not address the problems associated with the interference that is created through the use of ferromagnetic materials in the construction the patient support means that are currently used. The long procedure times, noise, and device and room heating are examples of the barriers associated with medical professionals and patients adopting the procedure.

Accordingly, one of skill will appreciate apparatuses and systems that address the above-mentioned problems that currently remain insufficiently addressed in the art of magnetic stimulation. Apparatuses and systems that provide a magnetic stimulation coil that can operate for sufficiently long periods of for each patient, can address several patients in series without stopping, and can perform within the allowed temperature limits for the magnetic stimulation coil would be appreciated by doctors and patients alike. Furthermore, apparatuses and systems that provide a combination of those features along with a great deal of flexibility in patient positioning and comfort, and do so using materials that do not interfere with the treatment, would provide a very strong contribution to the art.

### SUMMARY OF THE INVENTION

The teachings provided herein are generally directed to a system for delivering magnetic stimulation to a subject, wherein a force field generated by a magnet is applied to the subject for therapeutic purposes. In some embodiments, the teachings are directed to an apparatus for delivering magnetic stimulation to a subject, wherein the apparatus comprises a first magnetic stimulation coil, a power source, and a patient-positioning device. In these embodiments, the magnetic stimulation coil is (i) movably connected to patient-positioning device for fixably positioning the magnetic stimulation coil to stimulate a first region of a subject; and (ii) electrically connected to the power source. And, the patient positioning device provides an attachment site for a second magnetic stimulation coil, wherein the second magnetic stimulation coil is optionally movably connected to the patient-positioning device for positioning independently of the first magnetic stimulation coil to provide a combination delivery of magnetic stimulation to a second region of the subject.

The magnetic stimulation coil can comprise a transcranial magnetic stimulation coil and, in some embodiments, the patient positioning device supports the attachment of about 4 to about 10 independent transcranial magnetic stimulation coils. In some embodiments, the magnetic stimulation coil is removably connected to the patient-positioning device.

In some embodiments, the patient-positioning device can comprise a support surface that is adjustable to provide a desired hip flexion and knee flexion. The device can have the function of positioning the subject in a partially-recumbent position and adjustably supporting the subject's lower legs, upper legs, back, and head. In these embodiments, the device can comprise substantially non-ferromagnetic materials within a distance of about 2 feet or less from the position of the magnetic stimulation coil.

In some embodiments, the patient-positioning device comprises a reclining chair having a support for the subject's lower legs and a fully adjustable headrest. In these embodiments, the device can comprise substantially non-ferromagnetic materials within a distance of about 2 feet or less from the position of the magnetic stimulation coil.

In some embodiments, the patient-positioning device comprises a support surface that is adjustable to have the function of positioning the subject in an upright-sitting position, a partially-recumbent position, a fully-recumbent position, or a standing position. In these embodiments, the device can comprise substantially non-ferromagnetic materials within a distance of about 2 feet or less from the position of the magnetic stimulation coil.

In some embodiments, the patient-positioning device can comprise a headrest that is attached to an articulated arm, wherein both the headrest and the articulated arm are comprised of substantially non-ferromagnetic materials. In these embodiments, the device can comprise substantially non-ferromagnetic materials within a distance of about 2 feet or less from the position of the magnetic stimulation coil. And, the articulated arm can function to position and fix the headrest at a desired location in relation to the subject.

In some embodiments, the magnetic stimulation coil is connected to an articulated arm that is movably connected to the patient-positioning device, wherein the articulated arm is comprised of substantially non-ferromagnetic materials and functions to position and fix the magnetic stimulation coil at a desired location in relation to the subject.

The apparatus can have a combination of special features to provide more effective therapeutic conditions for a subject. In some embodiments, the teachings are directed to an apparatus for delivering transcranial magnetic stimulation to a subject, wherein the apparatus comprises a first transcranial magnetic stimulation coil, a power source, and a patient-positioning device. In these embodiments, the transcranial magnetic stimulation coil is (i) movably connected to patient-positioning device for fixably positioning the magnetic stimulation coil to stimulate a first cranial region of a subject; (ii) removably connected to the patient-positioning device through a first articulated arm, wherein the first articulated arm is comprised of substantially non-ferromagnetic materials and functions to position and fix the magnetic stimulation coil at a desired location in relation to the subject; and (iii) electrically connected to the power source.

Moreover, the patient positioning device provides an attachment site for an optional second transcranial magnetic stimulation coil, wherein the second transcranial magnetic stimulation coil is optionally movably connected to the patient-positioning device through an optional second articulated arm for positioning the second transcranial magnetic stimulation coil independently of the first transcranial magnetic stimulation coil. The combination of magnetic stimulation coils can be used to provide a combination delivery of transcranial magnetic stimulation to a second cranial region of the subject. The patient-positioning device can also comprise a first headrest that is attached to a third articulated arm, wherein both the first headrest and the third articulated arm are comprised of substantially non-ferromagnetic materials, and wherein the third articulated arm functions to position and fix the first headrest at a desired location to support the subject. And, the patient-positioning device can comprise an optional second headrest that is attached to an optional fourth articulated arm, wherein both the second headrest and the fourth articulated arm are comprised of substantially non-ferromagnetic materials, and wherein the fourth articulated arm functions to position and fix the second headrest at a desired location to support the subject. The patient-positioning device can also support the attachment of about 4 to about 10 independent transcranial magnetic stimulation coils.

In some embodiments, the teachings are directed to a system for delivering magnetic stimulation to a subject. In these embodiments, the system comprises a magnetic stimulation coil, a power source, and a patient-positioning device. The magnetic stimulation coil is (i) movably connected to patient-positioning device for fixably positioning the magnetic stimulation coil to stimulate a region of a subject and (ii) electrically connected to the power source. The system also comprises a cooling system for cooling the magnetic stimulation coil and maintaining the magnetic stimulation coil between a temperature of about 5° C. and about 40° C. during operation of the coil for a period of at least 90 minutes, wherein the system uses a cool gas to remove heat from the magnetic stimulation coil by direct convection. The patient positioning device can provide an attachment site for a second magnetic stimulation coil, wherein the second magnetic stimulation coil is optionally movably connected to the patient-positioning device for positioning the second magnetic stimulation coil independently of the first magnetic stimulation coil to provide a combination delivery of magnetic stimulation to a second region of the subject.

In some embodiments, the system further comprises a sound-resistant container containing the power source and the cooling system for cooling the magnetic stimulation coil. The sound-resistant container can also cool the power source, wherein the cooling system discharges cool air into the sound-resistant container. In some embodiments, the cooling system is a forced-air cooling system, and the forced-air is directed to cool the magnetic stimulation coil by convection. In some embodiments, the cooling system is a forced-air cooling system, and the forced-air is directed to cool the magnetic stimulation coil by convection and to cool the interior of the sound-resistant container. In some embodiments, a cooling system for cooling the magnetic stimulation coil maintains the magnetic stimulation coil between a temperature of about 5° C. and about 40° C. during operation of the coil for a period of at least 5 hours. And, in some embodiments, the magnetic stimulation coils comprise transcranial magnetic stimulation coils, and the patient-positioning device supports the attachment of about 4 to about 10 independent transcranial magnetic stimulation coils. The system described above can include any of the apparatuses described above.

In some embodiments, the teachings are directed to a magnetic stimulation coil assembly. The assembly includes a magnetic stimulation coil contained in a chamber within a housing. The housing has an inlet for a cool gas and an outlet for a heated gas. The assembly also includes a cooling system for producing the cool gas, a cool gas tube connected to the inlet for drawing the cool gas into the chamber and over the magnetic stimulation coil to cool the coil by direct convection during operation of the magnetic stimulation coil, and a heated gas tube connected to the outlet for drawing the heated gas away from the chamber. The assembly also includes a pump to create a pressure differential for moving the cool gas into the chamber and the heated gas out of the chamber.

Any of the apparatuses or systems described above can be used in a method of delivering a magnetic stimulation to a subject. In some embodiments, the method comprises fixably positioning the subject in the patient-positioning device, fixably positioning the first magnetic stimulation coil to stimulate the first region of the subject, selecting power settings at the power source to provide a desired first magnetic stimulation from the first magnetic stimulation coil, and treating the subject with the desired magnetic stimulation from the first magnetic stimulation coil. In some embodiments, the first magnetic stimulation coil is a transcranial magnetic stimulation coil, and the desired first magnetic stimulation is applied to a first cranial region of the subject. In some embodiments, the method further comprises fixably positioning the second magnetic stimulation coil to stimulate the second region of the subject, selecting power settings at the power source to provide a desired second magnetic stimulation from the second magnetic stimulation coil, and treating the subject with the desired second magnetic stimulation. In some embodiments, the first and second magnetic stimulation coils are transcranial stimulation coils, and the desired first and second magnetic stimulations are applied to first and second cranial regions of the subject.

### BRIEF DESCRIPTION OF THE DRAWINGS

FIG. 1 illustrates an apparatus for delivering magnetic stimulation to a subject according to some embodiments.

FIG. 2 illustrates a transcranial magnetic stimulation coil according to some embodiments.

FIGS. 3a and 3b illustrate a 3-D depiction of the magnetic field profile for a circular magnetic stimulation coil and a double circular magnetic stimulation coil.

FIG. 4 illustrates a patient-positioning device having a headrest attached to an articulated arm and a magnetic stimulation coil attached to an articulated arm according to some embodiments.

FIGS. 5a through 5c illustrate a patient-positioning device having the capacity for a plurality of patient positioning equipment components and transcranial magnetic stimulation coils attached to articulated arms. according to some embodiments.

FIGS. 6a and 6b illustrate a cooled magnetic stimulation system for delivering magnetic stimulation to a subject according to some embodiments.

FIGS. 7a and 7b illustrate a forced-air cooling system in a sound-resistant container according to some embodiments.

### DETAILED DESCRIPTION OF THE INVENTION

As described above, the teachings provided herein are generally directed to a system for delivering magnetic stimulation to a subject, wherein a force field generated by a magnet is applied to the subject for therapeutic purposes. As used herein, “transcranial magnetic stimulation” includes “repetitive transcranial magnetic stimulation.” Moreover, the term “subject” and “patient” can be used interchangeably in the present invention and refer to an animal such as a mammal including, but not limited to, non-primates such as, for example, a cow, pig, horse, cat, dog, rat and mouse; and primates such as, for example, a monkey or a human.

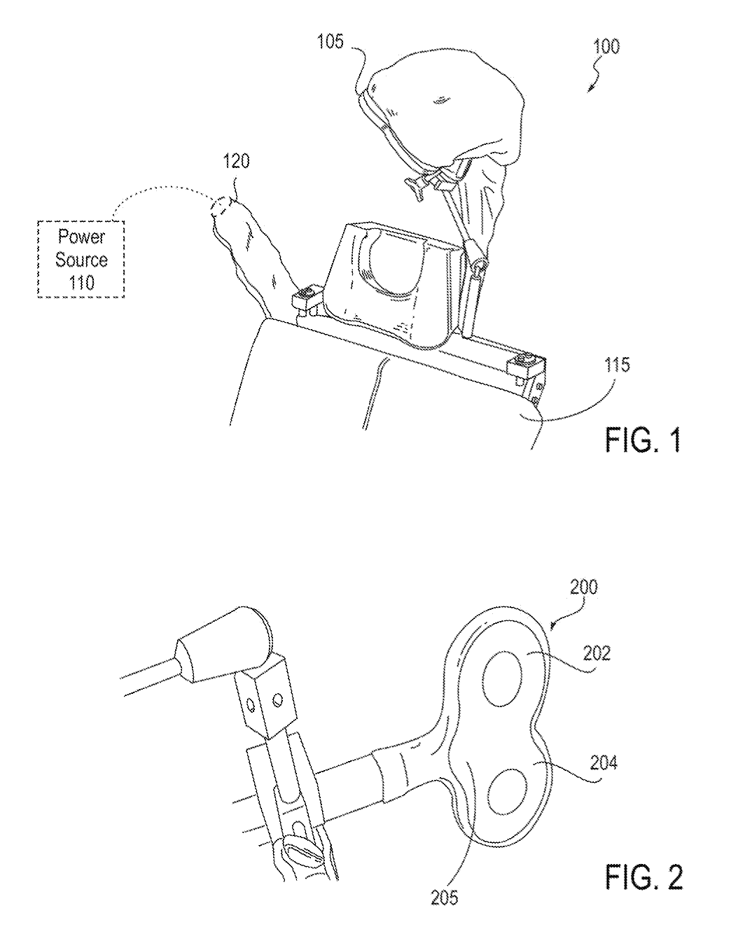

FIG. 1 illustrates an apparatus for delivering magnetic stimulation to a subject according to some embodiments. The apparatus 100 comprises a first magnetic stimulation coil 105, a power source 110, and a patient-positioning device 115. In these embodiments, the magnetic stimulation coil 105 is (i) movably connected to patient-positioning device 115 for fixably positioning the magnetic stimulation coil 105 to stimulate a first region of a subject; and (ii) electrically connected to the power source 110, using a power cable in conduit 120. And, the patient-positioning device 115 provides an attachment site for a second magnetic stimulation coil, wherein the second magnetic stimulation coil is optionally movably connected to the patient-positioning device 115 for positioning the second magnetic stimulation coil independently of the first magnetic stimulation coil 105 to provide a combination delivery of magnetic stimulation to a second region of the subject. A suitable power source and associated computation and control software for programming the power source can be obtained, for example, from The Magstim Company Ltd. of Wales, UK.

A “patient positioning device” can include any device known to one of skill for positioning a patient in a desired position for applying a therapy. In some embodiments, the patient positioning device can include a support surface for the patient's mandible to assist the patient in holding the patient's head in position for a treatment of an extended duration, such as a treatment period of more than a minute. In some embodiments, the patient-positioning device can include a chair, a bed, a circle bed, and the like. The choice of patient-positioning device, in many embodiments, is directed to the comfort of the patient to address the otherwise discomfort associated with the treatment of extended duration. In some embodiments the treatments can last for 30 minutes or longer in duration.

A component is “movably connected,” for example, when it can be connected in more than one location by merely moving the component from one to the other location. In some embodiments, the apparatuses and systems herein contain rails and movable brackets for movably connecting components to from one location to another. A component is “removably connected,” for example, when it can be connected a position, such as an otherwise fixed position, and readily removed for a particular purpose. In some embodiments, the apparatuses and systems herein contain articulated arms with a removable connection to a magnetic stimulation coil, wherein the user of the coil can readily remove the magnetic stimulation coil from an articulated arm for the particular purpose, for example, of locating a patient's motor threshold position and/or treatment position in magnetic stimulation therapy.

FIG. 2 illustrates a transcranial magnetic stimulation coil according to some embodiments. The transcranial magnetic stimulation coil 200 of FIG. 2 is the same magnetic stimulation coil 105 that is attached to the patient-positioning device 115 in the apparatus 100 of FIG. 1. The transcranial magnetic stimulation coil 200 is a double circular coil having two windings 202, 204 that are encased in the coil housing 205. Double circular transcranial magnetic stimulation coils can be obtained, for example, from the Magstim Company Ltd. of Wales, UK.

The selection of the power source and magnetic stimulation coil design will determine the type of magnetic field produced and used to stimulate a region of interest in or on the subject. In some embodiments, the magnetic field provided by the combination of the power source and the magnetic stimulation coil can be monophasic, biphasic, polyphasic magnetic fields, or a combination thereof, during a treatment. A monophasic magnetic field may be more accurate than a biphasic magnetic field and can also have lower noise and lower heat production; however, it may be more difficult to obtain a bilateral cortical response with a monophasic magnetic field. A biphasic magnetic field may have a short efficient pulse that is suited to a bilateral cortical stimulation; however it may have a higher noise and is possibly less accurate than the monophasic magnetic field. A polyphasic magnetic field may be more efficient and suited to bilateral cortical stimulation as well; however, it has the highest noise and heat production and is less accurate than a monophasic magnetic field.

One of skill can select the proper components for the apparatuses and systems taught herein to provide the desired type of magnetic field for a particular application. Tables 1 and 2 provide an example of design considerations that can be taken into account when designing an apparatus or system for a desired magnetic stimulation. Table 1, for example, lists physical characteristics and maximum calculated outputs of several different types of coils that are available.

TABLE 1

| | Circular 40 mm | Circular | Circular | Circular | Double Small | Double | Double | Double |
|---|---|---|---|---|---|---|---|---|
| | Type 9594 | 50 mm | 70 mm | 90 mm | 25 mm | 50 mm | 70 mm | Cone |
| | (discontinued) | Type 9993 | Type 9762 | Type 9784 | Type 1165 | Prototype | Type 9925 | Type 9902 |
| Inside Diameter (mm) | 20 | 25 | 40 | 66 | 18 (x2) | 34 (x2) | 56 (x2) | 96 (x2) |
| Outside Diameter (mm) | 62 | 77 | 94 | 123 | 42 (x2) | 74 (x2) | 87 (x2) | 125 (x2) |
| Number of Turns | 21 | 18 | 15 | 14 | 14 (x2) | 11 (x2) | 9 (x2) | 7 (x2) |
| Peak Magnetic Field | 4.1 | 3.6 | 2.6 | 2.0 | 4.6 | N/A | 2.2 | 1.4 |
| Strength (Tesla) | | | | | | | | |
| Peak Electric Field | 530 | 600 | 530 | 530 | 660 | N/A | 660 | N/A |
| Strength (Wm) | | | | | | | | |

Table 2 provides calculated figures of magnetic and electric field strengths, induced current, charge density, and deposited tissue energy for magnetic stimulation of the human brain. The calculations were performed for the purpose of considering the physiological effects of magnetic stimulation in humans, and it has been assumed that the magnetic coil is no closer than 5 mm to the brain in any of these measurements. Moreover, a uniform conductivity value of 0.35 S/m, that of grey matter in humans, was been used in these calculations of induced current, charge density per phase and energy deposited per pulse. The calculations are estimates that are designed to be the maximum possible value in each case and are likely overestimates. See Jalinous, R. Guide to Magnetic Stimulation, 24 (1998), available from The Magstim Company Ltd., Wales, UK.

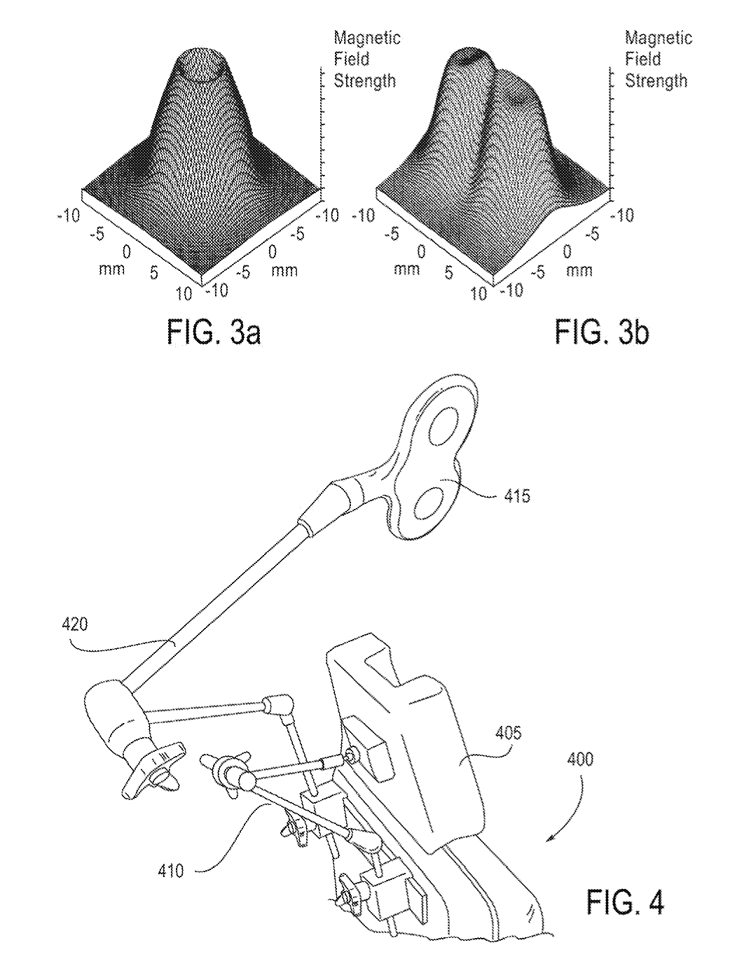

FIGS. 3a and 3b illustrate a 3-D depiction of the magnetic field profile for a circular magnetic stimulation coil and a double circular magnetic stimulation coil. FIG. 3a shows the profile of field strength produced by a circular magnetic stimulation coil using a 2.0 Tesla peak field. FIG. 3b shows the magnetic field strength profile produced by a double circular magnetic stimulation coil using a 2.2 Tesla peak field. This is an example of why one of skill will appreciate that the geometry and design of the magnetic stimulation coil will affect the magnetic stimulation that is achieved and, accordingly, will take such factors into account when designing an apparatus or system for a particular application.

In some embodiments, the patient positioning device supports the attachment of about 4 to about 10 independent transcranial magnetic stimulation coils. And, in some embodiments, the magnetic stimulation coil is removably connected to the patient-positioning device. The application of stimulation from multiple transcranial magnetic stimulation coils has found use in deep brain stimulation. Deep brain stimulation in select brain regions using electrical stimulation, for example, has provided remarkable therapeutic benefits for otherwise treatment-resistant movement and affective disorders such as chronic pain, Parkinson's disease, tremor, and dystonia.

In some embodiments, the patient-positioning device can comprise a support surface that is adjustable to provide a desired hip flexion and knee flexion. The device can have the function of positioning the subject in a partially-recumbent position and adjustably supporting the subject's lower legs, upper legs, back, and head. In these embodiments, the device can comprise substantially non-ferromagnetic materials within a distance of about 2 feet or less from the position of the magnetic stimulation coil.

“Non-ferromagnetic materials” can include, but are not limited to, titanium, aluminum, tin, copper, zinc, brass magnesium, ceramics, and polymers, wherein the choice of compositions comprising these materials does not display the property of ferromagnetism. However, one of skill will appreciate that there are also weakly ferromagnetic materials that may be suitable for the present teachings and include, but are not limited to, certain cobalt alloys, nickel alloys and austentic stainless steels which display a lesser degree of ferromagnetism than ferromagnetic materials. As such “substantially non-ferromagnetic materials” can include, for example, non-ferromagnetic and weakly ferromagnetic materials. Moreover, any material that does not provide unacceptable adverse interference to the magnetic stimulation treatment of a subject can be used with the present teachings. As such, the proximity of the material to the magnetic field is a parameter to consider when selecting materials to make the apparatuses and systems taught herein.

In some embodiments, the patient-positioning device comprises a reclining chair having a support for the subject's lower legs and a fully adjustable headrest. In these embodiments, the device can comprise substantially non-ferromagnetic materials within a distance of about 2 feet or less from the position of the magnetic stimulation coil.

In some embodiments, the patient-positioning device comprises a support surface that is adjustable to have the function of positioning the subject in an upright-sitting position, a partially-recumbent position, a fully-recumbent position, or a standing position. In these embodiments, the device can comprise substantially non-ferromagnetic materials within a distance of about 2 feet or less from the position of the magnetic stimulation coil.

FIG. 4 illustrates a patient-positioning device having a headrest attached to an articulated arm and a magnetic stimulation coil attached to an articulated arm according to some embodiments. As shown in FIG. 4, the patient-positioning device 400 can comprise a headrest 405 that is attached to an articulated arm 410, wherein both the headrest 405 and the articulated arm 410 are comprised of substantially non-ferromagnetic materials. And, the articulated arm 410 can function to position and fix the headrest 405 at a desired location in relation to the subject.

The headrest 405 can be placed in virtually any position in 3-dimensional space that is within the reach of the articulated arm 410, and the desired location of the headrest 405 can be selected for patient comfort, positioning of the magnetic coil, or a combination thereof. The magnetic stimulation coil 415 is connected to an articulated arm 420 that is movably connected to the patient-positioning device 400, wherein the articulated arm 420 is comprised of substantially non-ferromagnetic materials and functions to position and fix the magnetic stimulation coil 415 at a desired location in relation to the subject. In some embodiments, each of the articulated arms 410, 420 can include a single release lock that fixes each of the articulated arms 410, 420 in a desired position. Single release lock articulated arms, such as the FISSO articulated arm, can be obtained, for example, from Baitella AG of Zurich Switzerland.

In some embodiments, the headrest supports only the head and contacts the occipital region of the skull, supports the head and neck and contacts the occipital region of the skull and the upper cervical region of the neck, supports the head and contacts the frontal region of the skull and/or the mandible, or a combination thereof. In some embodiments, the headrest is conformal and conforms to the occipital region of the skull, one or both parietal regions of the skull, the frontal region of the skull, or a combination thereof. In many embodiments, the headrest comprises a soft, comfortable material that is non-ferromagnetic, such that the patient remains comfortable throughout a period of treatment. In some embodiments there are a plurality of headrests.

The apparatus can have a combination of features to provide more patient positioning equipment and magnetic stimulation coils to and thereby enhance the effectiveness of the therapeutic conditions for a subject.

Examples of patient positioning equipment include, but are not limited to, headrests, chinrests, cervical supports, and patient restraint equipment such as siderails and straps. In some embodiments, the teachings are directed to an apparatus for delivering transcranial magnetic stimulation to a subject, wherein the apparatus comprises a first transcranial magnetic stimulation coil, a power source, and a patient-positioning device. In these embodiments, the transcranial magnetic stimulation coil is (i) movably connected to patient-positioning device for fixably positioning the magnetic stimulation coil to stimulate a first cranial region of a subject; (ii) removably connected to the patient-positioning device through a first articulated arm, wherein the first articulated arm is comprised of substantially non-ferromagnetic materials and functions to position and fix the magnetic stimulation coil at a desired location in relation to the subject; and (iii) electrically connected to the power source.

Moreover, the patient positioning device can provide an attachment site for an optional second transcranial magnetic stimulation coil, wherein the second transcranial magnetic stimulation coil is optionally movably connected to the patient-positioning device through an optional second articulated arm for positioning the second transcranial magnetic stimulation coil independently of the first transcranial magnetic stimulation coil. The combination of magnetic stimulation coils can be used to provide a combination delivery of transcranial magnetic stimulation to a second cranial region of the subject. The patient-positioning device can also comprise a first headrest that is attached to a third articulated arm, wherein both the first headrest and the third articulated arm are comprised of substantially non-ferromagnetic materials, and wherein the third articulated arm functions to position and fix the first headrest at a desired location to support the subject. And, the patient-positioning device can comprise an optional second headrest, for example, that is attached to an optional fourth articulated arm, wherein both the second headrest and the fourth articulated arm are comprised of substantially non-ferromagnetic materials, and wherein the fourth articulated arm functions to position and fix the second headrest at a desired location to support the subject. The patient-positioning device can also support the attachment of about 4 to about 10 independent transcranial magnetic stimulation coils. In some embodiments, the patient-positioning device can also support the attachment of about 4 to about 10 independent transcranial magnetic stimulation coils and 4 headrests.

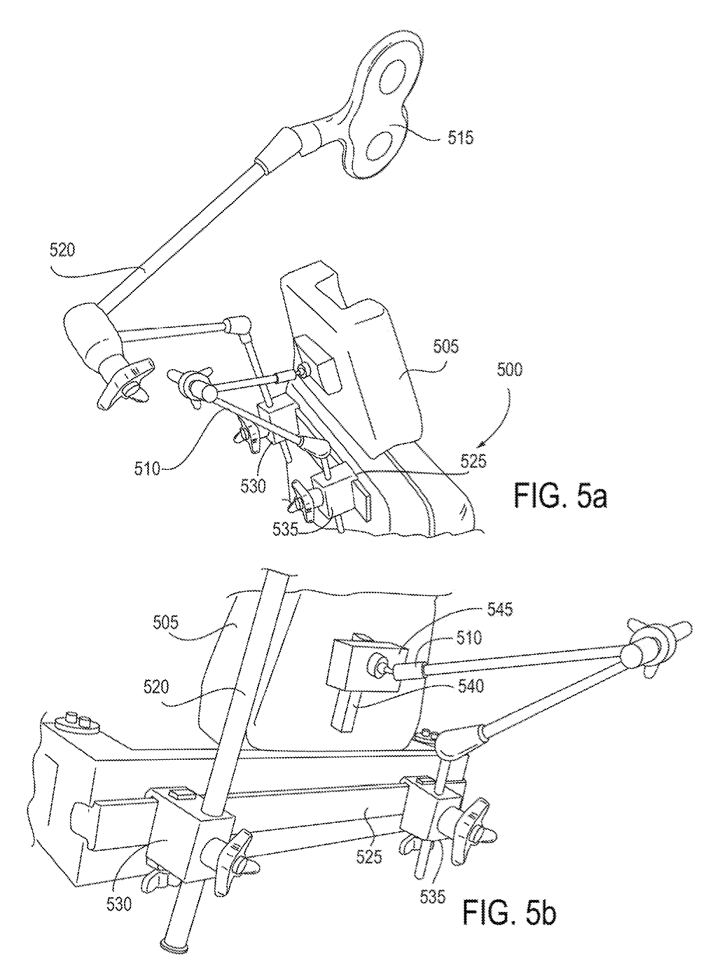

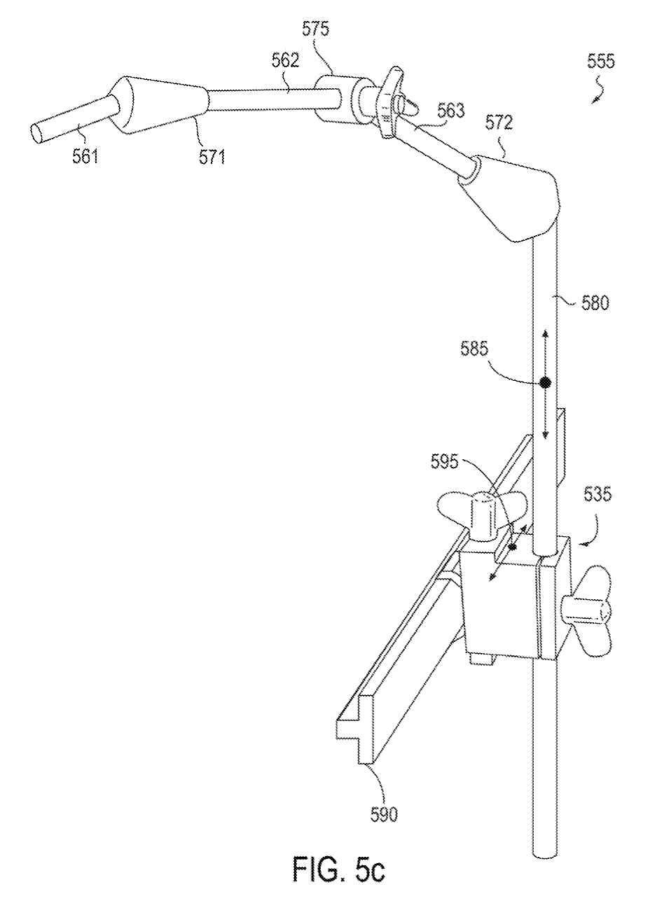

FIGS. 5a through 5c illustrate a patient-positioning device having the capacity for a plurality of patient positioning equipment components and transcranial magnetic stimulation coils attached to articulated arms. according to some embodiments. FIG. 5a shows that the patient-positioning device 500 can comprise a headrest 505 that is attached to a first articulated arm 510, wherein both the headrest 505 and the first articulated arm 510 are comprised of substantially non-ferromagnetic materials. And, the first articulated arm 510 can function to position and fix the headrest 505 at a desired location in relation to the subject.

The headrest 505 can be placed in virtually any position desired in 3-dimensional space that is within the reach of the first articulated arm 510, and the desired location of the headrest 505 can be selected for patient comfort, positioning of the magnetic coil, or a combination thereof. The magnetic stimulation coil 515 is connected to a second articulated arm 520 that is movably connected to the patient-positioning device 500, wherein the second articulated arm 520 is comprised of substantially non-ferromagnetic materials and functions to position and fix the magnetic stimulation coil 515 at a desired location in relation to the subject.

FIG. 5b shows a means of attaching a plurality of transcranial magnetic stimulation coils and patient positioning equipment, such as those described above. A rail 525 has substantial free space for slidably attaching and fixing additional clamps such as the clamps 530, 535, that are used to secure the first articulated arm 510 and second articulated arm 520. The additional claims can be used to secure additional articulated arms that are used to position and fix additional patient positioning equipment and magnetic stimulation coils. In FIG. 5b, headrest 505 is slidably attached to headrest rail 540 and headrest claim 545 for additional flexibility in patient positioning.

FIG. 5c illustrates an articulated arm that may be used to provide an ease of full range of motion and adjustment, according to some embodiments. The articulated arm 555 is a single release articulated arm, meaning that single release mechanism 575 is the locking means that needs to be activated or deactivated to free or fix the all of the motion at the articulations 571,572 of the articulated arm 555.

The articulated arm 555 has at least three rigid arms 561,562,563 that are linkably connected to the articulations 571,572 and the single release mechanism 575. The articulations 571,572 comprise ball joints that are both simultaneously released or locket by the single release mechanism 575. The rigid arms 561,562,563 and articulations 571,572 are linkably connected to a column 580, which is slidably attached to a rail 590 using a releasable bracket, such as the bracket 535 (or 530) as shown in FIGS. 5a and 5b. Through releasable bracket 535, the column 580 can slide vertically 585 or horizontally 595, which when combined with the extremely wide range of mobility of the articulations 571,572, the total mobility of the articulated arm 555 gives one of skill a full range of motion to fixably position a magnetic stimulation coil, or any patient positioning equipment, such as a headrest, in virtually any position in the 3-dimensional space around a patient that is within reach of the articulated arm 555. One of skill will appreciate that the rails can be positioned, and attached to, around any form of patient-positioning device in virtually any location to facility positioning and fixation of a multitude of magnetic coils in nearly any position in the three-dimensional space surrounding a patient.

One of skill will also appreciate an apparatus or system that can consistently maintain the temperature of the magnetic stimulation coils within allowed temperature limits for a period of time that substantially exceeds the operating time limits associated with current systems, and these periods of time should not vary considerable with geographic location. Current magnetic stimulation units use ambient air for example, which has a temperature and moisture content that varies by location, so performance of these systems will also vary considerably by location, making the operating limits somewhat indeterminate.

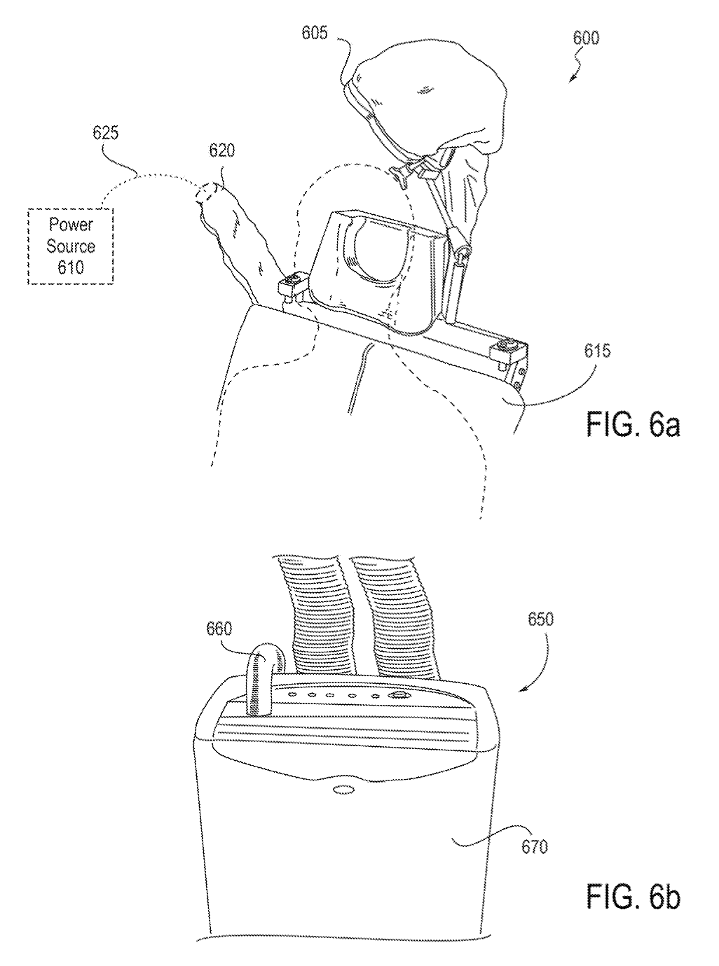

FIGS. 6a and 6b illustrate a cooled magnetic stimulation system for delivering magnetic stimulation to a subject according to some embodiments. FIG. 6a shows that the system 600 comprises a magnetic stimulation coil 605, a power source 610, and a patient-positioning device 615. The magnetic stimulation coil 605 is (i) movably connected to patient-positioning device 615 for fixably positioning the magnetic stimulation coil 605 to stimulate a region of a subject and (ii) electrically connected to the power source 610 using a power cable 625 in conduit 620.

FIG. 6b shows a cooling system 650 for cooling the magnetic stimulation coil 605 and permitting operation of the magnetic stimulation coil 605 for a period of greater than, in some embodiments, about 1 hour, 2 hours, 3 hours, 4 hours, 5 hours, 6 hours, 12 hours, 18 hours, and even 24 hours, without a substantial loss of performance. The cooling system 650 cools the magnetic stimulation coil by delivering forced cooled air through an air conditioning hose 660 in conduit 620 that pass the forced cooled air produced by air conditioning unit 670 over the internal coils in magnetic stimulation coil 605. In some embodiments, the system can maintain the magnetic stimulation coil between a temperature of about 5° C. and about 40° C. during operation of the coil for a period of at least 90 minutes, wherein the system uses a cool gas to remove heat from the magnetic stimulation coil by direct convection. In some embodiments, where a plurality of coils are used, each coil can be cooled, independently, or together, using the same or additional cooling systems.

The sound produced during transcranial magnetic stimulation, for example, can be uncomfortable to the patient. Discharge “click noise” depends on the coil size, where small coils are louder than large coils, and also depends on the power level and manufacturing method of the coil. It has been suggested, for example, that the discharge click noise produced by a 5 cm stimulating coil causes hearing loss in albino and chinchilla rabbits when the coil is placed over the external auditory opening. In fact, the sound was measured to be as much as 157 dB peak SPL. Standard commercial coils have been found to operate at a maximum of 124 dB(A) on the coil surface and fall to 117 dB(A) when about 50 mm away from the coil surface. Accordingly, since a substantial component in the effective treatment of a patient can include making the patient comfortable, reducing the noise associated with the operation of the apparatuses and systems taught herein can certainly help to accomplish that objective. In some embodiments, the system further comprises a sound-resistant container for housing containing the noisy components of the magnetic stimulation systems taught herein, such as the power source and/or the cooling system for cooling the magnetic stimulation coil.

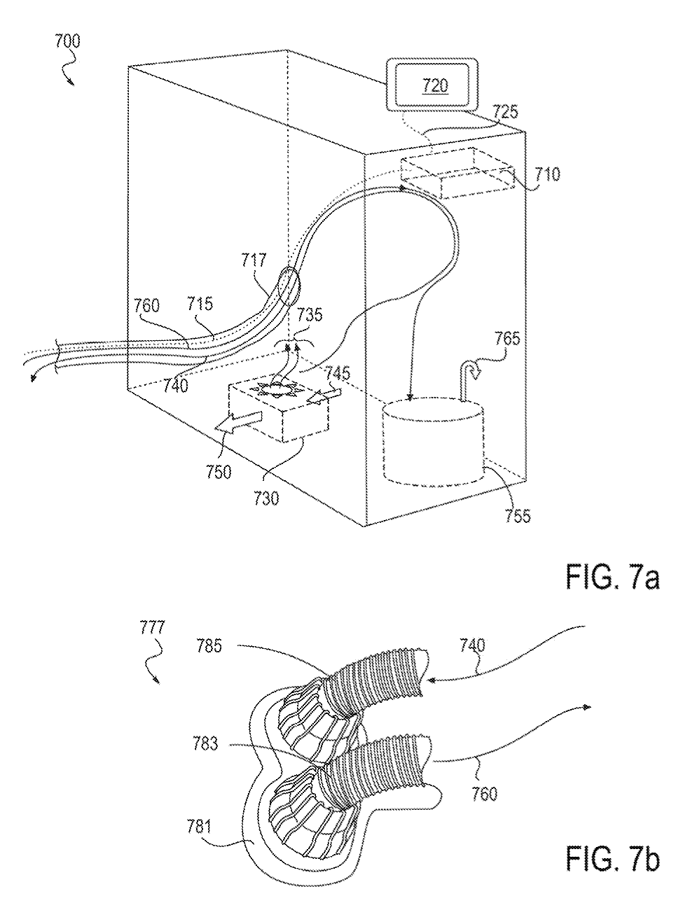

FIGS. 7a and 7b illustrate a forced-air cooling system in a sound-resistant container according to some embodiments. In FIG. 7a, the sound-resistant container 700 houses the forced-air cooling system 730,755 and the power source 710. The power source 710 is connected to a controller/user-interface 720 by interface cable 725 and is connected to the magnetic stimulation coil assembly 777 by power cable 715 and enclosed in conduit 717. Cool air 735 is produced by a heat engine 730, also known as a cooling system, and the cool air 735 is drawn as a cool stream 740 from heat engine 730 through a tube 785 in conduit 717 and into to a chamber inside housing 781 that houses the magnetic stimulation coil. The cooling stream 740 is drawn using pump 755, wherein pump 755 is also connected through a tube 783 in conduit 717 to the chamber inside housing 781 that houses the magnetic coil, such that the cooling stream 740 is drawn by the pump 755 over the magnetic stimulation coil to cool the coil by direct convection, and directed into the pump 755. The action of drawing the cooling stream 740 over the magnetic coil creates a heated stream 760, and pump 755 discharges the heated stream 760 into the interior of the sound-resistant container 700.

The heated stream 760 is discharged as spent air 765 into the space in the sound-resistant container and allowed to mix with the ambient air 745. The ambient air is drawn into heat engine 730 and cooled to produce the cool air 735 and cooling stream 740. The heat engine 730 discharges heat as heated air 750, and this heated air 750 can be discharged into the environment surrounding the exterior of the sound-resistant container 700, or it can be discharged outside of the building to avoid heat accumulation in the treatment area. The cooling air 740 is directed to cool the magnetic stimulation coil using a direct convection heat transfer process. The cool air 735 is directed to cool the interior of the sound-resistant container and, in some embodiments, also provides a desired dehumidification. Accordingly, the sound-resistant container can cool the components, such as the power source, which are contained by the container.

In some embodiments, direct convention can come from a gas, such that the cooling system is a forced gas cooling system, where the gas can include, but is not limited to air, inert gases, carbon dioxide, and any combination thereof. The apparatus and systems taught herein can consistently operate at 120% motor threshold at 10 MHz, and can operate for at least 60 minutes, 90 minutes, 2 hours, 3 hours, 5 hours, 12 hours, 18 hours, or 24 hours and stay within the allowed temperature limits of 5° C. to 40° C. It should be appreciated that the heat produced varies due to the chosen design. Temperatures that are induced vary among monophasic, biphasic, or polyphasic magnetic field systems. The allowed temperature limits can range from about 5° C. to about 40° C., from about 3° C. to about 60° C., from about 10° C. to about 50° C. from about 5° C. to about 30° C., from about 15° C. to about 25° C., from about 20° C. to about 35° C., or any range therein.

In some embodiments, the cooling systems taught herein can allow one of skill to operate the magnetic simulation coil for a period of greater than about 24 hours without a substantial loss of performance. The amount of heat produced by the system is proportion to the extent of the coil operation and number of coils. In some embodiments, for example, the patient-positioning device supports the attachment of about 4 to about 10 independent coils, such as transcranial magnetic stimulation coils. The system described above can include any of the apparatuses described above.

In some embodiments, the magnetic stimulation coil can be in an assembly that can be used in a variety of apparatuses and systems. The assembly can comprise a magnetic stimulation coil contained in a chamber within a housing having an inlet for a cool gas and an outlet for a heated gas. A cooling system is needed for producing the cool gas. The cool gas is drawn through a cool gas tube that is connected to the inlet, where the cool gas enters the chamber and cools the magnetic stimulation coil through direct convection during operation of the magnetic stimulation coil. And, the heated gas that is produced during the direct convection is drawn away from the magnetic stimulation coil through a heated gas tube that is connected to the outlet. A pump is included to induce the pressure differential needed to move the cool gas and the heated gas into and out of the chamber.

Any of the apparatuses or systems described above can be used in a method of delivering a magnetic stimulation to a subject. In some embodiments, the method comprises fixably positioning the subject in the patient-positioning device, fixably positioning the first magnetic stimulation coil to stimulate the first region of the subject, selecting power settings at the power source to provide a desired first magnetic stimulation from the first magnetic stimulation coil, and treating the subject with the desired magnetic stimulation from the first magnetic stimulation coil. In some embodiments, the first magnetic stimulation coil is a transcranial magnetic stimulation coil, and the desired first magnetic stimulation is applied to a first cranial region of the subject.

“Fixably positioning the subject” refers to positioning the subject such that the subject can receive the magnetic stimulation treatment for an extended period of time such as, for example, for a period ranging from about 30 seconds to about 3 hours, from about 3 minutes to about 30 minutes, from about 10 minutes to about 1 hour, or any range therein. These are long periods for which a patient can be expected to remain still for a particular treatment, and even longer periods of treatment may be contemplated. Accordingly, comfort can become a very important parameter in the treatment of the patient.

In some embodiments, a subject may be fixably positioned, for example by providing a comfortable setting that includes a soft support surface for the patient, additional patient-positioning equipment, such as the soft, fully adjustable headrests taught herein, and perhaps a second headrest, arm or leg supports, and the like. In some embodiments, the subject may be infirm, and a comfortable setting may not be enough to fixably position the subject, such that a restraint of some sort, such as a soft strap or a second headrest, may be added to help fix the subject in the desired position.

In some cases, the patient's position may require special care due to an existing injury, such that the patient would rather be placed in an unusual position, such as an oblique or tilted position, to ensure that further injury or discomfort does not occur. One of skill will appreciate that fixably positioning the subject can be accomplished using several known methods including, but not limited to, uncomfortable techniques such as fixing the subject in place using a headpiece and uncomfortable cranial screws, a method which is undesired in most embodiments. In some embodiments, a plurality of patient positioning equipment can be used, such as four independent headrests, for example, that can fixably position a patient in a vegetative state, and the positioning can be comfortable for the patient. A plurality of patient positioning equipment can include support for the front of the head, chin, and both sides of the head; or in some embodiments, can include support for the chin and the back of the neck, for example.

In some embodiments, the method further comprises fixably positioning the second magnetic stimulation coil to stimulate the second region of the subject, selecting power settings at the power source to provide a desired second magnetic stimulation from the second magnetic stimulation coil, and treating the subject with the desired second magnetic stimulation. In some embodiments, the first and second magnetic stimulation coils are transcranial stimulation coils, and the desired first and second magnetic stimulations are applied to first and second cranial regions of the subject.

Each of the magnetic stimulations described herein can be administered to a subject in combination therapy. The delivery of the magnetic stimulation can be administered, for example, concomitantly, sequentially, or cyclically to a subject. In some embodiments, cycling therapy involves, for example, the administering a first magnetic stimulation for a predetermined period of time, administering a second magnetic stimulation for a second predetermined period of time, and repeating this cycling for any desired purpose such as, for example, to enhance the efficacy of the treatment or, perhaps to deepen the penetration of the stimulation, such as in the case of deep brain stimulation. The magnetic stimulations of the present invention can also be administered concurrently. The term “concurrently” is not limited to the administration at exactly the same time, but rather means that the magnetic stimulations can be administered in a sequence and time interval such that the magnetic stimulations can work together to provide additional benefit. Each magnetic stimulation can be administered separately or together in any appropriate combination of intensity, sequence, or cycle, where the combinations are any combination that could be contemplated by one of skill.

In some embodiments, for example the magnetic stimulations can be administered at points in time that vary by about 0.05 to about 0.5 seconds, from about 0.1 seconds to about 1.0 second, from about 1.0 minute to about 5 minutes, from about 5 minutes to about 15 minutes, from about 15 minutes to about 30 minutes, from about 30 minutes to 1 hour, from about 1 hour to about 2 hours, from about 2 hours to about 4 hours, from about 4 hours to about 8 hours, from about 8 hours to about 12 hours, or any range therein. The stimulations can be repeated every 12 hours, every 18 hours, every 24 hours, every 48 hours, weekly, bi-weekly, bi-monthly, or monthly, for example.

### EXAMPLE 1

In the treatment of depression, a MAGSTIM RAPID² repetitive Transcranial Magnetic Stimulator (rTMS) device is used, along with the magnetic stimulating coil assembly, apparatus, and system improvements taught herein, particularly as shown in the FIGS. 1-7.

The magnetic stimulation parameters used in the treatments is as follows:
Apply the stimulus to the left prefrontal cortex region of the brain;
Use 110% power of motor threshold;
Use 10 Hz;
Use 4-second trains (each train includes 40 pulses at 10 Hz);
Use 26 seconds between trains (30 seconds between the start of one train and the start of the following train);
Apply therapy in 40 minute sessions (actually 37 minutes and 4 seconds, including 3000 pulses in 75 trains);
Apply therapy 5 days/week for 4-6 weeks; and
Apply therapy in maintenance sessions once per week after remission is achieved.

Approximately one-third of patients treated have seen complete resolution of depression, about another one-third saw a significant improvement, and the final one-third saw no significant improvement. Of all patients, 75% experienced scalp pain during the first few treatments, 50% experienced facial pain during the first few treatments, and 25% experienced a tension headache during the first few treatments. Other side effects included mania in bipolar patients (common), high frequency hearing loss (rare), and seizure (rare).

### EXAMPLE 2

The temperature of the air conditioning cooling system was tested for performance in the system described Example 1 and FIGS. 1-7. The temperature at which air conditioning unit is set will depend on the surrounding environment of the apparatus and system. A hotter ambient temperature will require a lower setting, for example.

A 10,000 BTU consumer grade air conditioner was used and was set at a variety of ranges to accommodate the average temperature of Palo Alto, Calif., which has average temperatures ranging from about 60° F. to about 80° F.:

Temperatures ranging from about 60° F. to about 75° F. were used and assessed for their ability to cool the biphasic magnetic stimulation coil taught in Example 1. It was discovered that a setting of 75° F. was insufficient for proper cooling and 70° F. was adequate, but a setting of 61° F. provided the best results, and it was assumed that the improvement was at least partially based on the dehumidification of the air that occurred in the air conditioning system at the lower temperature setting.

Those skilled in the art will recognize, or be able to ascertain using no more than routine experimentation, that there are many equivalents to the specific embodiments described herein. Such equivalents are intended to be encompassed by the following claims. All publications, patents, and patent applications mentioned in this application are herein incorporated by reference into the specification to the same extent as if each was specifically indicate to be herein incorporated by reference in its entirety.

We claim:

1. An apparatus for delivering magnetic stimulation to a subject, wherein the apparatus comprises:
a first magnetic stimulation coil, a power source, and a patient-positioning device; wherein,
the magnetic stimulation coil is (i) movably connected to patient-positioning device for fixably positioning the magnetic stimulation coil to stimulate a first region of a subject; and (ii) electrically connected to the power source; and,
the patient positioning device provides an attachment site for a second magnetic stimulation coil, wherein the second magnetic stimulation coil is optionally movably connected to the patient-positioning device for positioning independently of the first magnetic stimulation coil to provide a combination delivery of magnetic stimulation to a second region of the subject.

2. The apparatus of claim 1, wherein the magnetic stimulation coil comprises a transcranial magnetic stimulation coil.

3. The apparatus of claim 2, wherein the patient positioning device supports the attachment of about 4 to about 10 independent transcranial magnetic stimulation coils.

4. The apparatus of claim 1, wherein the magnetic stimulation coil is removably connected to the patient-positioning device.

5. The apparatus of claim 1, wherein the patient-positioning device comprises a support surface that is adjustable to provide a desired hip flexion and knee flexion, wherein the device has the function of positioning the subject in a partially-recumbent position and adjustably supporting the subject's lower legs, upper legs, back, and head; and the device comprises substantially non-ferromagnetic materials within a distance of about 2 feet or less from the position of the magnetic stimulation coil.

6. The apparatus of claim 1, wherein the patient-positioning device comprises a reclining chair having a support for the subject's lower legs and a fully adjustable headrest, and the device comprises substantially non-ferromagnetic materials within a distance of about 2 feet or less from the position of the magnetic stimulation coil.

7. The apparatus of claim 1, wherein the patient-positioning device comprises a support surface that is adjustable to have the function of positioning the subject in an upright-sitting position, a partially-recumbent position, a fully-recumbent position, or a standing position, and the device comprises substantially non-ferromagnetic materials within a distance of about 2 feet or less from the position of the magnetic stimulation coil.

8. The apparatus of claim 1, wherein the patient-positioning device comprises a headrest that is attached to an articulated arm, wherein both the headrest and the articulated arm are comprised of substantially non-ferromagnetic materials, and the device comprises substantially non-ferromagnetic materials within a distance of about 2 feet or less from the position of the magnetic stimulation coil, and wherein the articulated arm functions to position and fix the headrest at a desired location in relation to the subject.

9. The apparatus of claim 1, wherein the magnetic stimulation coil is connected to an articulated arm that is movably connected to the patient-positioning device, wherein the articulated arm is comprised of substantially non-ferromagnetic materials and functions to position and fix the magnetic stimulation coil at a desired location in relation to the subject.

10. An apparatus for delivering transcranial magnetic stimulation to a subject, wherein the apparatus comprises:
a first transcranial magnetic stimulation coil, a power source, and a patient-positioning device; wherein,
the transcranial magnetic stimulation coil is (i) movably connected to patient-positioning device for fixably positioning the magnetic stimulation coil to stimulate a first cranial region of a subject; (ii) removably connected to the patient-positioning device through a first articulated arm, wherein the first articulated arm is comprised of substantially non-ferromagnetic materials and functions to position and fix the magnetic stimulation coil at a desired location in relation to the subject; and (iii) electrically connected to the power source;
the patient positioning device provides an attachment site for an optional second transcranial magnetic stimulation coil, wherein the second transcranial magnetic stimulation coil is optionally movably connected to the patient-positioning device through an optional second articulated arm for positioning the second transcranial magnetic stimulation coil independently of the first transcranial magnetic stimulation coil to provide a combination delivery of transcranial magnetic stimulation to a second cranial region of the subject;
the patient-positioning device comprises a first headrest that is attached to a third articulated arm, wherein both the first headrest and the third articulated arm are comprised of substantially non-ferromagnetic materials, and wherein the third articulated arm functions to position and fix the first headrest at a desired location to support the subject; and
the patient-positioning device comprises an optional second headrest that is attached to an optional fourth articulated arm, wherein both the second headrest and the fourth articulated arm are comprised of substantially non-ferromagnetic materials, and wherein the fourth articulated arm functions to position and fix the second headrest at a desired location to support the subject.

11. The apparatus of claim 10, wherein the patient-positioning device supports the attachment of about 4 to about 10 independent transcranial magnetic stimulation coils.

12. A system for delivering magnetic stimulation to a subject, wherein the system comprises:
a magnetic stimulation coil, a power source, and a patient-positioning device; wherein, the magnetic stimulation coil is (i) movably connected to patient-positioning device for fixably positioning the magnetic stimulation coil to stimulate a region of a subject and (ii) electrically connected to the power source; and,
a cooling system for cooling the magnetic stimulation coil and maintaining the magnetic stimulation coil between a temperature of about 5° C. and about 40° C. during operation of the coil for a period of at least 90 minutes, wherein the system uses a cool gas to remove heat from the magnetic stimulation coil by direct convection.

13. The system of claim 12, wherein the patient positioning device provides an attachment site for a second magnetic stimulation coil, wherein the second magnetic stimulation coil is optionally movably connected to the patient-positioning device for positioning the second magnetic stimulation coil independently of the first magnetic stimulation coil to provide a combination delivery of magnetic stimulation to a second region of the subject.

14. The system of claim 12, wherein the system further comprises a sound-resistant container containing the power source and the cooling system for cooling the magnetic stimulation coil, the power source, or a combination thereof.

15. The system of claim 12, wherein the cooling system is a forced-air cooling system, and the forced-air is directed to cool the magnetic stimulation coil by convection.

16. The system of claim 12, wherein the cooling system is a forced-air cooling system, and the forced-air is directed to cool the magnetic stimulation coil by convection and to cool the interior of the sound-resistant container.

17. The system of claim 12, wherein the cooling system maintains the magnetic stimulation coil between a temperature of about 5° C. and about 40° C. during operation of the coil for a period of at least 5 hours.

18. The system of claim 12, wherein the magnetic stimulation coils comprise transcranial magnetic stimulation coils, and the patient-positioning device supports the attachment of about 4 to about 10 independent transcranial magnetic stimulation coils.

19. A system for delivering transcranial magnetic stimulation to a subject, wherein the system comprises:
the apparatus of claim 10;
a cooling system for cooling the transcranial magnetic stimulation coil and maintaining the magnetic stimulation coil between a temperature of about 5° C. and about 40° C. during operation of the coil for a period of at least 90 minutes, wherein the system uses a cool gas to remove heat from the magnetic stimulation coil by direct convection; and
a sound-resistant container containing the power source and the cooling system for cooling the transcranial magnetic stimulation coil, the power source, or a combination thereof.

20. The system of claim 19, wherein the cooling system is a forced-air cooling system, and the forced-air is directed to cool the transcranial magnetic stimulation coil by convection and to cool the interior of the sound-resistant container.

21. The system of claim 19, wherein the cooling system maintains the magnetic stimulation coil between a temperature of about 5° C. and about 40° C. during operation of the coil for a period of at least 5 hours.

22. A magnetic stimulation coil assembly comprising:
a magnetic stimulation coil contained in a chamber within a housing, wherein the housing has an inlet for a cool gas and an outlet for a heated gas;
a cooling system for producing the cool gas;
a cool gas tube connected to the inlet for drawing the cool gas into the chamber and over the magnetic stimulation coil to cool the coil by direct convection during operation of the magnetic stimulation coil;
a heated gas tube connected to the outlet for drawing the heated gas away from the chamber; and
a pump to create a pressure differential for moving the cool gas into the chamber and the heated gas out of the chamber.

23. A method of delivering a magnetic stimulation to a subject comprising using the apparatus of claim 1, wherein the using comprises:
fixably positioning the subject in the patient-positioning device;
fixably positioning the first magnetic stimulation coil to stimulate the first region of the subject;
selecting power settings at the power source to provide a desired first magnetic stimulation from the first magnetic stimulation coil; and
treating the subject with the desired magnetic stimulation from the first magnetic stimulation coil.

24. The method of claim 23, wherein the first magnetic stimulation coil is a transcranial magnetic stimulation coil, and the desired first magnetic stimulation is applied to a first cranial region of the subject.

25. The method of claim 23, wherein the method further comprises fixably positioning the second magnetic stimulation coil to stimulate the second region of the subject, selecting power settings at the power source to provide a desired second magnetic stimulation from the second magnetic stimulation coil, and treating the subject with the desired second magnetic stimulation.

26. A method of delivering a transcranial magnetic stimulation to a subject comprising using the apparatus of claim 3, wherein the using comprises:
fixably positioning the subject in the patient-positioning device;
fixably positioning the magnetic stimulation coils to provide at cranial stimulation to the subject;
selecting power settings at the power source to provide a desired magnetic stimulation from each of the transcranial magnetic stimulation coils; and
treating the subject with the desired magnetic stimulations from each of the transcranial magnetic stimulation coils.

27. A method of delivering a magnetic stimulation to a subject using the apparatus of claim 10, wherein the method comprises:
fixably positioning the subject in the patient-positioning device, wherein fixably positioning includes fixably positioning the first headrest;
fixably positioning the first magnetic stimulation coil to stimulate the first region of the subject;
selecting power settings at the power source to provide a desired first magnetic stimulation from the first magnetic stimulation coil; and
treating the subject with the desired magnetic stimulation from the first magnetic stimulation coil.

28. The method of claim 27, wherein the first magnetic stimulation coil is a transcranial magnetic stimulation coil, and the desired first magnetic stimulation is applied to a first cranial region of the subject.

29. A method of delivering a magnetic stimulation to a subject comprising using the system of claim 12, wherein the using comprises:
fixably positioning the subject in the patient-positioning device;
fixably positioning the first magnetic stimulation coil to stimulate the first region of the subject;
selecting power settings at the power source to provide a desired first magnetic stimulation from the first magnetic stimulation coil; and
treating the subject with the desired magnetic stimulation from the first magnetic stimulation coil.

30. A method of delivering a magnetic stimulation to a subject comprising using the system of claim 19, wherein the using comprises:
fixably positioning the subject in the patient-positioning device, wherein fixably positioning includes fixably positioning the first headrest;
fixably positioning the first magnetic stimulation coil to stimulate the first region of the subject;
selecting power settings at the power source to provide a desired first magnetic stimulation from the first magnetic stimulation coil; and
treating the subject with the desired magnetic stimulation from the first magnetic stimulation coil.
